(19) United States
(12) Patent Application Publication
(10) Pub. No.: US 2010/0222217 A1
(43) Pub. Date: Sep. 2, 2010

(54) Fungicidal N-[2-(Haloalkoxy)Phenyl]Heteroarylcarboxamides

(76) Inventors: Markus Gewehr, Kastellaun (DE); Jochen Dietz, Mannheim (DE); Thomas Grote, Wachenheim (DE); Wassilios Grammenos, Ludwigshafen (DE); Bernd Müller, Frankenthal (DE); Jan Klaas Lohmann, Mannheim (DE)

Correspondence Address: BIRCH STEWART KOLASCH & BIRCH, PO BOX 747, FALLS CHURCH, VA, 22040-0747, US

(21) Appl. No.: 11/989,973

(22) PCT Filed: Aug. 2, 2006
(86) PCT No.: PCT/EP2006/064991

§ 371 Date: Feb. 4, 2008

(30) Foreign Application Priority Data
Aug. 5, 2005 (DE) 102005037679.7
Aug. 16, 2005 (DE) 102005038964.3
Aug. 22, 2005 (DE) 102005039785.9

(51) Int. Cl. A01N 25/26; C07D 231/40; A01N 43/56; C07D 277/46; A01N 43/78; C07D 213/75; A61K 31/44; A01P 3/00

(52) U.S. Cl. 504/100; 548/372.5; 514/404; 548/195; 514/371; 546/309; 514/352

(57) ABSTRACT

N-[2-(Haloalkoxy)phenyl]heteroarylcarboxamides of the formula I, where n=0 or 1, Hal=halogen, X=C₂-C₄-haloalkyl, Het=a pyrazole, thiazole or pyridine radical (a), (b) or (c), where R¹=C₁-C₄-alkyl or C₁-C₄-haloalkyl, R²=hydrogen or halogen, R³=C₁-C₄-alkyl or C₁-C₄-haloalkyl and R⁵=halogen, C₁-C₄-alkyl or C₁-C₄-haloalkyl; except for N-[2-(1,1,2,2-tetrafluoroethoxy)phenyl]-1,3-dimethylpyrazol-4-yl-carboxamide, N-[2-(1,1,2,2-tetrafluoroethoxy)phenyl]-3-trifluoromethyl-1-methylpyrazol-4-yl-carboxamide and N-[2-(2,2,2-trifluorethoxy)phenyl]-3-trifluormethyl-1-methylpyrazol-4-yl-carboxamide. A fungicidal composition comprising at least one compound I, the use of the compounds I for preparing a composition suitable for controlling harmful fungi, a method for controlling harmful fungi using the compounds I and also seed comprising at least one compound I.

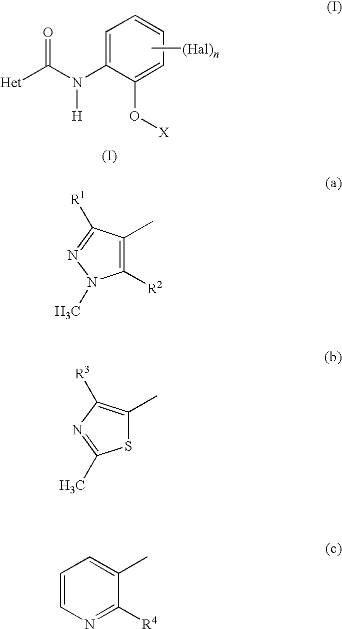

The present invention relates to N[2-(haloalkoxy)phenyl]heteroarylcarboxamides of the formula I

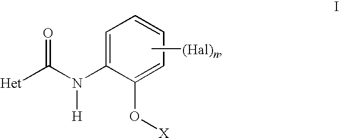

in which the variables are as defined below:
n is 0 or 1;
Hal is halogen;
X is C₂-C₄-haloalkyl;
Het is a pyrazole, thiazole or pyridine radical of the formula (a), (b) or (c)

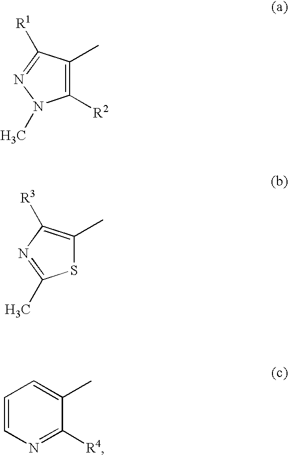

where
R¹ is C₁-C₄-alkyl or C₁-C₄-haloalkyl,
R² is hydrogen or halogen,
R³ is C₁-C₄-alkyl or C₁-C₄-haloalkyl and
R⁴ is halogen, C₁-C₄-alkyl or C₁-C₄-haloalkyl,

except for N-[2-(1,1,2,2-tetrafluoroethoxy)phenyl]-1,3-dimethylpyrazol-4-yl-carboxamide, N-[2-(1,1,2,2-tetrafluoroethoxy)phenyl]-3-trifluoromethyl-1-methylpyrazol-4-yl-carboxamide and N-[2-(2,2,2-trifluoroethoxy)phenyl]-3-trifluoromethyl-1-methylpyrazol-4-yl-carboxamide.

Moreover, the invention relates to processes for preparing these compounds, to compositions comprising them and to their use for controlling phytopathogenic harmful fungi.

N-[2-(1,1,2,2-Tetrafluoroethoxy)phenyl]-1,3-dimethylpyrazol-4-ylcarboxamide and N-[2-(1,1,2,2-tetrafluoroethoxy)phenyl]-3-trifluoromethyl-1-methylpyrazol-4-ylcarboxamide and their fungicidal action are already known from EP-A 589301.

N-[2-(1,1,2,2-tetrafluoroethoxy)phenyl]-3-trifluoromethyl-1-methylpyrazol-4-yl-carboxamide and N-[2-(2,2,2-trifluoroethoxy)phenyl]-3-trifluoromethyl-1-methylpyrazol-4-yl-carboxamide and their fungicidal action are known from JP 04/316559.

Moreover, compounds of the type of the N-[2-(haloalkoxy)phenyl]heteroarylcarboxamides I are also described in EP-A 545099, U.S. Pat. No. 7,015,218, JP 10/072420, JP 63/048269 and JP 2001/342179. However, the fungicidal activity of the known compounds is not always entirely satisfactory.

Accordingly, it was an object of the present invention to provide novel compounds from this structural class having improved fungicidal activity and/or an improved activity spectrum.

We have found that this object is achieved by the N-[2-(haloalkoxy)phenyl]heteroarylcarboxamides defined at the outset. Furthermore, we have found compositions comprising them and methods for controlling harmful fungi using the compounds I.

Compared to the known compounds, the compounds of the formula I have an improved activity against harmful fungi.

The compounds of the formula I may be present in various crystal modifications which may differ in their biological activity. They also form part of the subject matter of the present invention.

In formula I, halogen is fluorine, chlorine, bromine or iodine, preferably fluorine or chlorine;

C₁-C₄-alkyl is methyl, ethyl, n-propyl, 1-methylethyl, n-butyl, 1-methylpropyl, 2-methylpropyl or 1,1-dimethylethyl, preferably methyl or ethyl;

C₁-C₄-haloalkyl is a partially or fully halogenated C₁-C₄-alkyl radical, where the halogen atom(s) is/are in particular fluorine, chlorine and/or bromine, i.e., for example, chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloro-ethyl, 1-bromoethyl, 1-fluoroethyl, 2-fluoroethyl, 2,2-difluoroethyl, 2-chloro-2-fluoroethyl, 2,2,2-trifluoroethyl, 2-chloro-1,1,2-trifluoroethyl, 2-chloro-2,2-difluoroethyl, 2-bromo-2,2-difluoroethyl, 2,2-dichloro-2-fluoroethyl, 2,2,2-trichloroethyl, 1,1,2,2-tetrafluoroethyl, 1,1,2,2-tetrachloroethyl, pentafluoroethyl, 2,2,3,3-tetrafluoro-1-propyl, 1,1,2,3,3,3-hexa-fluoro-1-propyl, 1,1,1,3,3,3-hexafluoro-2-propyl, heptafluoro-1-propyl, heptafluoro-2-propyl, 2,2,3,3,4,4,4-heptafluoro-1-butyl or nonafluoro-1-butyl, in particular halomethyl, particularly preferably CH₂—Cl, CH(Cl)₂, CH₂—F, CH(F)₂, CF₃, CHFCl, CF₂Cl or CF(Cl)₂;

C₂-C₄-haloalkyl is a partially or fully halogenated C₂-C₄-alkyl radical, where the halogen atom(s) is/are in particular fluorine and, if desired, chlorine, i.e., for example, 1-fluoroethyl, 2-fluoroethyl, 2,2-difluoroethyl, 2-chloro-2-fluoroethyl, 2,2,2-trifluoroethyl, 2-chloro-1,1,2-trifluoroethyl, 2-chloro-2,2-difluoroethyl, 1,1,2,2-tetrafluoroethyl, pentafluoroethyl, 2,2,3,3-tetrafluoro-1-propyl, 1,1,2,3,3,3-hexafluoro-1-propyl, 1,1,1,3,3,3-hexafluoro-2-propyl, heptafluoro-1-propyl, heptafluoro-2-propyl, 2,2,3,3,4,4,4-hepta-fluoro-1-butyl or nonafluoro-1-butyl,

in particular ethyl which carries 1 to 5 halogen atoms selected from a group consisting of 5 fluorine atoms and 1 chlorine atom,

or is n-propyl which carries 1 to 7 halogen atoms selected from a group consisting of 7

fluorine atoms and 1 chlorine atom,

or is isopropyl which carries 1 to 7 halogen atoms selected from a group consisting of 7 fluorine atoms and 1 chlorine atom.

With respect to the intended use of the N-[2-(haloalkoxy)phenyl]heteroarylcarboxamides of the formula I, particular preference is given to the following meanings of the substituents, in each case on their own or in combination:
n is zero;
Hal is fluorine or chlorine, in particular fluorine;
X is 1,1,2,2-tetrafluoroethyl, 2-chloro-1,1,2-trifluoroethyl or 1,1,2,3,3,3-hexafluoropropyl, in particular 2-chloro-1,1,2-trifluoroethyl or 1,1,2,3,3,3-hexafluoropropyl.

Moreover, preference is given to those compounds I in which Het is a pyrazole radical of the formula (a).

Preference is given to compound I where Het=(a) and X is C₃-C₄-haloalkyl or, if R¹ is C₂-C₄-alkyl, CHF₂ or CHFCl, X may also be C₂-haloalkyl.

Among the compounds I where Het=(a), very particular preference is given to those where R¹=methyl or difluoromethyl, in particular difluoromethyl, and/or R²=halogen, in particular fluorine or chlorine.

Preference is also given to compounds I in which Het is a thiazole radical of the formula (b).

Preference is furthermore given to those compounds I in which Het is a pyridine radical of the formula (c).

Among the N-[2-(haloalk(enyl)oxy)phenyl]carboxamides I in which Het is a pyrazole radical (a), preference is given to those compounds in which R¹ is methyl or halomethyl, in particular CH₃, CHF₂ or CF₃.

Among the compounds I where Het=(a) and R¹=trifluoromethyl, very particular preference is given to those where X=2-chloro-1,1,2-trifluoroethyl or 1,1,2,3,3,3-hexafluoropropyl.

Among the compounds I where Het=(a) and R¹=methyl or difluoromethyl, very particular preference is given to those where X=1,1,2,2-tetrafluoroethyl.

Preference is furthermore given to compounds I where Het=(a) in which R² is hydrogen, fluorine or chlorine. Preference is also given here, firstly, to those compounds I where R²=hydrogen, and also, secondly, to those compounds I where R²=fluorine or chlorine, in particular fluorine.

Among the compounds I where Het=(a) and R²=hydrogen, very particular preference is given to those where X=2-chloro-1,1,2-trifluoroethyl or 1,1,2,3,3,3-hexafluoropropyl. Among the compounds I where Het=(a) and R²=fluorine or chlorine, very particular preference is given to those where X=1,1,2,2-tetrafluoroethyl.

Among the N-[2-(haloalk(enyl)oxy)phenyl]carboxamides I in which Het is a thiazole radical (b), preference is given to those compounds in which R³ is methyl or halo-methyl, in particular CH₃, CHF₂ or CF₃.

Among the N-[2-(haloalk(enyl)oxy)phenyl]carboxamides I in which Het is a pyridine radical (c), preference is given to those compounds in which R⁴ is halogen, methyl or halomethyl, in particular fluorine, chlorine, bromine, methyl, CHF₂ or CF₃.

Particular preference is given to the compounds Ia, Ib and Ic listed in Tables 1, 2 and 3 below.

TABLE 1
Compounds Ia [I where n = 0 and Het = a
pyrazole radical of the formula (a)]

| Compound | | | |
|---|---|---|---|
| Number | R¹ | R² | X |
| Ia.1 | CH₃ | H | CH₂—CHF₂ |
| Ia.2 | CH₃ | H | CH₂—CF₃ |
| Ia.3 | CH₃ | H | CF₂—CF₃ |
| Ia.4 | CH₃ | H | CF₂—CHF—Cl |
| Ia.5 | CH₃ | H | CF₂—CHF—CF₃ |
| Ia.6 | CH₃ | H | CH(CF₃)₂ |
| Ia.7 | CH₃ | H | CH₂—CF₂—CF₃ |
| Ia.8 | CH₃ | H | CH₂—CF₂—CHF₂ |
| Ia.9 | CH₃ | H | CH₂—CHF—CHF₂ |
| Ia.10 | CH₃ | H | CH₂—CHF—CF₃ |
| Ia.11 | CH₃ | H | CF₂—CF₂—CF₃ |
| Ia.12 | CH₃ | H | CF(CF₃)₂ |
| Ia.13 | CH₃ | H | CF₂—CF₂—CF₂—CF₃ |
| Ia.14 | CH₃ | F | CH₂—CHF₂ |
| Ia.15 | CH₃ | F | CH₂—CF₃ |
| Ia.16 | CH₃ | F | CF₂—CHF₂ |
| Ia.17 | CH₃ | F | CF₂—CF₃ |
| Ia.18 | CH₃ | F | CF₂—CHF—Cl |
| Ia.19 | CH₃ | F | CF₂—CHF—CF₃ |
| Ia.20 | CH₃ | F | CH(CF₃)₂ |
| Ia.21 | CH₃ | F | CH₂—CF₂—CF₃ |
| Ia.22 | CH₃ | F | CH₂—CF₂—CHF₂ |
| Ia.23 | CH₃ | F | CH₂—CHF—CHF₂ |
| Ia.24 | CH₃ | F | CH₂—CHF—CF₃ |
| Ia.25 | CH₃ | F | CF₂—CF₂—CF₃ |
| Ia.26 | CH₃ | F | CF(CF₃)₂ |
| Ia.27 | CH₃ | F | CF₂—CF₂—CF₂—CF₃ |
| Ia.28 | CH₃ | Cl | CH₂—CHF₂ |
| Ia.29 | CH₃ | Cl | CH₂—CF₃ |
| Ia.30 | CH₃ | Cl | CF₂—CHF₂ |
| Ia.31 | CH₃ | Cl | CF₂—CF₃ |
| Ia.32 | CH₃ | Cl | CF₂—CHF—Cl |
| Ia.33 | CH₃ | Cl | CF₂—CHF—CF₃ |
| Ia.34 | CH₃ | Cl | CH(CF₃)₂ |
| Ia.35 | CH₃ | Cl | CH₂—CF₂—CF₃ |
| Ia.36 | CH₃ | Cl | CH₂—CF₂—CHF₂ |
| Ia.37 | CH₃ | Cl | CH₂—CHF—CHF₂ |
| Ia.38 | CH₃ | Cl | CH₂—CHF—CF₃ |
| Ia.39 | CH₃ | Cl | CF₂—CF₂—CF₃ |
| Ia.40 | CH₃ | Cl | CF(CF₃)₂ |
| Ia.41 | CH₃ | Cl | CF₂—CF₂—CF₂—CF₃ |
| Ia.42 | CHF₂ | H | CH₂—CHF₂ |
| Ia.43 | CHF₂ | H | CH₂—CF₃ |
| Ia.44 | CHF₂ | H | CF₂—CHF₂ |
| Ia.45 | CHF₂ | H | CF₂—CF₃ |
| Ia.46 | CHF₂ | H | CF₂—CHF—Cl |
| Ia.47 | CHF₂ | H | CF₂—CHF—CF₃ |
| Ia.48 | CHF₂ | H | CH(CF₃)₂ |
| Ia.49 | CHF₂ | H | CH₂—CF₂—CF₃ |
| Ia.50 | CHF₂ | H | CH₂—CF₂—CHF₂ |
| Ia.51 | CHF₂ | H | CH₂—CHF—CHF₂ |
| Ia.52 | CHF₂ | H | CH₂—CHF—CF₃ |
| Ia.53 | CHF₂ | H | CF₂—CF₂—CF₃ |
| Ia.54 | CHF₂ | H | CF(CF₃)₂ |
| Ia.55 | CHF₂ | H | CF₂—CF₂—CF₂—CF₃ |
| Ia.56 | CHF₂ | F | CH₂—CHF₂ |
| Ia.57 | CHF₂ | F | CH₂—CF₃ |
| Ia.58 | CHF₂ | F | CF₂—CHF₂ |
| Ia.59 | CHF₂ | F | CF₂—CF₃ |
| Ia.60 | CHF₂ | F | CF₂—CHF—Cl |
| Ia.61 | CHF₂ | F | CF₂—CHF—CF₃ |
| Ia.62 | CHF₂ | F | CH(CF₃)₂ |
| Ia.63 | CHF₂ | F | CH₂—CF₂—CF₃ |
| Ia.64 | CHF₂ | F | CH₂—CF₂—CHF₂ |
| Ia.65 | CHF₂ | F | CH₂—CHF—CHF₂ |
| Ia.66 | CHF₂ | F | CH₂—CHF—CF₃ |
| Ia.67 | CHF₂ | F | CF₂—CF₂—CF₃ |
| Ia.68 | CHF₂ | F | CF(CF₃)₂ |
| Ia.69 | CHF₂ | F | CF₂—CF₂—CF₂—CF₃ |
| Ia.70 | CHF₂ | Cl | CH₂—CHF₂ |
| Ia.71 | CHF₂ | Cl | CH₂—CF₃ |
| Ia.72 | CHF₂ | Cl | CF₂—CHF₂ |
| Ia.73 | CHF₂ | Cl | CF₂—CF₃ |
| Ia.74 | CHF₂ | Cl | CF₂—CHF—Cl |
| Ia.75 | CHF₂ | Cl | CF₂—CHF—CF₃ |
| Ia.76 | CHF₂ | Cl | CH(CF₃)₂ |
| Ia.77 | CHF₂ | Cl | CH₂—CF₂—CF₃ |
| Ia.78 | CHF₂ | Cl | CH₂—CF₂—CHF₂ |
| Ia.79 | CHF₂ | Cl | CH₂—CHF—CHF₂ |
| Ia.80 | CHF₂ | Cl | CH₂—CHF—CF₃ |
| Ia.81 | CHF₂ | Cl | CF₂—CF₂—CF₃ |
| Ia.82 | CHF₂ | Cl | CF(CF₃)₂ |
| Ia.83 | CHF₂ | Cl | CF₂—CF₂—CF₂—CF₃ |
| Ia.84 | CF₃ | H | CH₂—CHF₂ |
| Ia.85 | CF₃ | H | CH₂—CF₃ |
| Ia.86 | CF₃ | H | CF₂—CF₃ |
| Ia.87 | CF₃ | H | CF₂—CHF—Cl |
| Ia.88 | CF₃ | H | CF₂—CHF—CF₃ |
| Ia.89 | CF₃ | H | CH(CF₃)₂ |
| Ia.90 | CF₃ | H | CH₂—CF₂—CF₃ |
| Ia.91 | CF₃ | H | CH₂—CF₂—CHF₂ |
| Ia.92 | CF₃ | H | CH₂—CHF—CHF₂ |
| Ia.93 | CF₃ | H | CH₂—CHF—CF₃ |
| Ia.94 | CF₃ | H | CF₂—CF₂—CF₃ |
| Ia.95 | CF₃ | H | CF(CF₃)₂ |
| Ia.96 | CF₃ | H | CF₂—CF₂—CF₂—CF₃ |
| Ia.97 | CF₃ | F | CH₂—CHF₂ |
| Ia.98 | CF₃ | F | CH₂—CF₃ |
| Ia.99 | CF₃ | F | CF₂—CHF₂ |
| Ia.100 | CF₃ | F | CF₂—CF₃ |
| Ia.101 | CF₃ | F | CF₂—CHF—Cl |
| Ia.102 | CF₃ | F | CF₂—CHF—CF₃ |
| Ia.103 | CF₃ | F | CH(CF₃)₂ |
| Ia.104 | CF₃ | F | CH₂—CF₂—CF₃ |
| Ia.105 | CF₃ | F | CH₂—CF₂—CHF₂ |
| Ia.106 | CF₃ | F | CH₂—CHF—CHF₂ |
| Ia.107 | CF₃ | F | CH₂—CHF—CF₃ |
| Ia.108 | CF₃ | F | CF₂—CF₂—CF₃ |
| Ia.109 | CF₃ | F | CF(CF₃)₂ |
| Ia.110 | CF₃ | F | CF₂—CF₂—CF₂—CF₃ |
| Ia.111 | CF₃ | Cl | CH₂—CHF₂ |
| Ia.112 | CF₃ | Cl | CH₂—CF₃ |
| Ia.113 | CF₃ | Cl | CF₂—CHF₂ |
| Ia.114 | CF₃ | Cl | CF₂—CF₃ |
| Ia.115 | CF₃ | Cl | CF₂—CHF—Cl |
| Ia.116 | CF₃ | Cl | CF₂—CHF—CF₃ |
| Ia.117 | CF₃ | Cl | CH(CF₃)₂ |
| Ia.118 | CF₃ | Cl | CH₂—CF₂—CF₃ |
| Ia.119 | CF₃ | Cl | CH₂—CF₂—CHF₂ |
| Ia.120 | CF₃ | Cl | CH₂—CHF—CHF₂ |
| Ia.121 | CF₃ | Cl | CH₂—CHF—CF₃ |
| Ia.122 | CF₃ | Cl | CF₂—CF₂—CF₃ |
| Ia.123 | CF₃ | Cl | CF(CF₃)₂ |
| Ia.124 | CF₃ | Cl | CF₂—CF₂—CF₂—CF₃ |

TABLE 2
Compounds Ib [I where n = 0, R⁴ = CH₃ and
Het = a thiazole radical of the formula (b)]

| Compound | | |
|---|---|---|
| Number | R³ | X |
| Ib.1 | CH₃ | CH₂—CHF₂ |
| Ib.2 | CH₃ | CH₂—CF₃ |
| Ib.3 | CH₃ | CF₂—CHF₂ |
| Ib.4 | CH₃ | CF₂—CF₃ |
| Ib.5 | CH₃ | CF₂—CHF—Cl |
| Ib.6 | CH₃ | CF₂—CHF—CF₃ |
| Ib.7 | CH₃ | CH(CF₃)₂ |
| Ib.8 | CH₃ | CH₂—CF₂—CF₃ |
| Ib.9 | CH₃ | CH₂—CF₂—CHF₂ |
| Ib.10 | CH₃ | CH₂—CHF—CHF₂ |
| Ib.11 | CH₃ | CH₂—CHF—CF₃ |
| Ib.12 | CH₃ | CF₂—CF₂—CF₃ |
| Ib.13 | CH₃ | CF(CF₃)₂ |
| Ib.14 | CH₃ | CF₂—CF₂—CF₂—CF₃ |
| Ib.15 | CHF₂ | CH₂—CHF₂ |
| Ib.16 | CHF₂ | CH₂—CF₃ |
| Ib.17 | CHF₂ | CF₂—CHF₂ |
| Ib.18 | CHF₂ | CF₂—CF₃ |
| Ib.19 | CHF₂ | CF₂—CHF—Cl |
| Ib.20 | CHF₂ | CF₂—CHF—CF₃ |
| Ib.21 | CHF₂ | CH(CF₃)₂ |
| Ib.22 | CHF₂ | CH₂—CF₂—CF₃ |
| Ib.23 | CHF₂ | CH₂—CF₂—CHF₂ |
| Ib.24 | CHF₂ | CH₂—CHF—CHF₂ |
| Ib.25 | CHF₂ | CH₂—CHF—CF₃ |
| Ib.26 | CHF₂ | CF₂—CF₂—CF₃ |
| Ib.27 | CHF₂ | CF(CF₃)₂ |
| Ib.28 | CHF₂ | CF₂—CF₂—CF₂—CF₃ |
| Ib.29 | CF₃ | CH₂—CHF₂ |
| Ib.30 | CF₃ | CH₂—CF₃ |
| Ib.31 | CF₃ | CF₂—CHF₂ |
| Ib.32 | CF₃ | CF₂—CF₃ |
| Ib.33 | CF₃ | CF₂—CHF—Cl |
| Ib.34 | CF₃ | CF₂—CHF—CF₃ |
| Ib.35 | CF₃ | CH(CF₃)₂ |
| Ib.36 | CF₃ | CH₂—CF₂—CF₃ |
| Ib.37 | CF₃ | CH₂—CF₂—CHF₂ |
| Ib.38 | CF₃ | CH₂—CHF—CHF₂ |
| Ib.39 | CF₃ | CH₂—CHF—CF₃ |
| Ib.40 | CF₃ | CF₂—CF₂—CF₃ |
| Ib.41 | CF₃ | CF(CF₃)₂ |
| Ib.42 | CF₃ | CF₂—CF₂—CF₂—CF₃ |

TABLE 3
Compounds Ic [I where n = 0 and
Het = a pyridine radical of the formula (c)]

| Compound | | |
|---|---|---|
| Number | R⁴ | X |
| Ic.1 | CH₃ | CH₂—CHF₂ |
| Ic.2 | CH₃ | CH₂—CF₃ |
| Ic.3 | CH₃ | CF₂—CHF₂ |
| Ic.4 | CH₃ | CF₂—CF₃ |
| Ic.5 | CH₃ | CF₂—CHF—Cl |
| Ic.6 | CH₃ | CF₂—CHF—CF₃ |
| Ic.7 | CH₃ | CH(CF₃)₂ |
| Ic.8 | CH₃ | CH₂—CF₂—CF₃ |
| Ic.9 | CH₃ | CH₂—CF₂—CHF₂ |
| Ic.10 | CH₃ | CH₂—CHF—CHF₂ |
| Ic.11 | CH₃ | CH₂—CHF—CF₃ |
| Ic.12 | CH₃ | CF₂—CF₂—CF₃ |
| Ic.13 | CH₃ | CF(CF₃)₂ |
| Ic.14 | CH₃ | CF₂—CF₂—CF₂—CF₃ |
| Ic.15 | CHF₂ | CH₂—CHF₂ |
| Ic.16 | CHF₂ | CH₂—CF₃ |
| Ic.17 | CHF₂ | CF₂—CHF₂ |
| Ic.18 | CHF₂ | CF₂—CF₃ |
| Ic.19 | CHF₂ | CF₂—CHF—Cl |
| Ic.20 | CHF₂ | CF₂—CHF—CF₃ |
| Ic.21 | CHF₂ | CH(CF₃)₂ |
| Ic.22 | CHF₂ | CH₂—CF₂—CF₃ |
| Ic.23 | CHF₂ | CH₂—CF₂—CHF₂ |
| Ic.24 | CHF₂ | CH₂—CHF—CHF₂ |
| Ic.25 | CHF₂ | CH₂—CHF—CF₃ |
| Ic.26 | CHF₂ | CF₂—CF₂—CF₃ |
| Ic.27 | CHF₂ | CF(CF₃)₂ |
| Ic.28 | CHF₂ | CF₂—CF₂—CF₂—CF₃ |
| Ic.29 | CF₃ | CH₂—CHF₂ |
| Ic.30 | CF₃ | CH₂—CF₃ |
| Ic.31 | CF₃ | CF₂—CHF₂ |
| Ic.32 | CF₃ | CF₂—CF₃ |
| Ic.33 | CF₃ | CF₂—CHF—Cl |
| Ic.34 | CF₃ | CF₂—CHF—CF₃ |
| Ic.35 | CF₃ | CH(CF₃)₂ |
| Ic.36 | CF₃ | CH₂—CF₂—CF₃ |
| Ic.37 | CF₃ | CH₂—CF₂—CHF₂ |
| Ic.38 | CF₃ | CH₂—CHF—CHF₂ |
| Ic.39 | CF₃ | CH₂—CHF—CF₃ |
| Ic.40 | CF₃ | CF₂—CF₂—CF₃ |
| Ic.41 | CF₃ | CF(CF₃)₂ |
| Ic.42 | CF₃ | CF₂—CF₂—CF₂—CF₃ |
| Ic.43 | F | CH₂—CHF₂ |
| Ic.44 | F | CH₂—CF₃ |
| Ic.45 | F | CF₂—CHF₂ |
| Ic.46 | F | CF₂—CF₃ |
| Ic.47 | F | CF₂—CHF—Cl |
| Ic.48 | F | CF₂—CHF—CF₃ |
| Ic.49 | F | CH(CF₃)₂ |
| Ic.50 | F | CH₂—CF₂—CF₃ |
| Ic.51 | F | CH₂—CF₂—CHF₂ |
| Ic.52 | F | CH₂—CHF—CHF₂ |
| Ic.53 | F | CH₂—CHF—CF₃ |
| Ic.54 | F | CF₂—CF₂—CF₃ |
| Ic.55 | F | CF(CF₃)₂ |
| Ic.56 | F | CF₂—CF₂—CF₂—CF₃ |
| Ic.57 | Cl | CH₂—CHF₂ |
| Ic.58 | Cl | CH₂—CF₃ |
| Ic.59 | Cl | CF₂—CHF₂ |
| Ic.60 | Cl | CF₂—CF₃ |
| Ic.61 | Cl | CF₂—CHF—Cl |
| Ic.62 | Cl | CF₂—CHF—CF₃ |
| Ic.63 | Cl | CH(CF₃)₂ |
| Ic.64 | Cl | CH₂—CF₂—CF₃ |
| Ic.65 | Cl | CH₂—CF₂—CHF₂ |
| Ic.66 | Cl | CH₂—CHF—CHF₂ |
| Ic.67 | Cl | CH₂—CHF—CF₃ |
| Ic.68 | Cl | CF₂—CF₂—CF₃ |
| Ic.69 | Cl | CF(CF₃)₂ |
| Ic.70 | Cl | CF₂—CF₂—CF₂—CF₃ |

N-8 2-(1,1,2,2-Tetrafluoroethoxy)phenyl]-3-difluoromethyl-1-methylpyrazol-4-yl-carboxamide,

N-[2-(1,1,2,2-tetrafluoroethoxy)phenyl]-1,3-dimethyl-5-fluoropyrazol-4-ylcarboxamide,

N-[2-(1,1,2,2-tetrafluoroethoxy)phenyl]-3-difluoromethyl-5-fluoro-1-methylpyrazol-4-ylcarboxamide,

N-[2-(1,1,2,2-tetrafluoroethoxy)phenyl]-5-fluoro-1-methyl-3-trifluoromethylpyrazol-4-ylcarboxamide,

N-[2-(1,1,2,2-tetrafluoroethoxy)phenyl]-5-chloro-1,3-dimethylpyrazol-5-chloro-4-ylcarboxamide,

N-[2-(1,1,2,2-tetrafluoroethoxy)phenyl]-5-chloro-3-difluoromethyl-1-methylpyrazol-4-ylcarboxamide,

N-[2-(1,1,2,2-tetrafluoroethoxy)phenyl]-5-chloro-1-methyl-3-trifluoromethylpyrazol-4-ylcarboxamide,

N-[2-(1,1,2,2-tetrafluoroethoxy)phenyl]-2,4-dimethylthiazol-5-ylcarboxamide,

N-[2-(1,1,2,2-tetrafluoroethoxy)phenyl]-4-difluoromethyl-2-methylthiazol-5-yl-carboxamide,

N-[2-(1,1,2,2-tetrafluoroethoxy)phenyl]-2-methyl-4-trifluoromethylthiazol-5-yl-carboxamide,

N-[2-(1,1,2,2-tetrafluoroethoxy)phenyl]-2-methylnicotinamide,

N-[2-(1,1,2,2-tetrafluoroethoxy)phenyl]-2-trifluoromethylnicotinamide,

N-[2-(1,1,2,2-tetrafluoroethoxy)phenyl]-2-fluoronicotinamide,

N-[2-(1,1,2,2-tetrafluoroethoxy)phenyl]-2-chloronicotinamide,

N-[2-(2-chloro-1,1,2-trifluoroethoxy)phenyl]-2-chloronicotinamide,

N-[2-(2-chloro-1,1,2-trifluoroethoxy)phenyl]-2-methyl-4-trifluoromethylthiazole-5-carboxamide,

N-[2-(1,1,2,2-tetrafluoroethoxy)phenyl]-3-(chlorofluoromethyl)-1-methyl-1H-pyrazole-4-carboxamide,

N-[2-(1,1,2,2-tetrafluoroethoxy)phenyl]-3-fluoromethyl-1-methyl-1H-pyrazole-4-carboxamide,

N-[2-(1,1,2,2-tetrafluoroethoxy)phenyl]-3-(dichlorofluoromethyl)-1-methyl-1H-pyrazole-4-carboxamide,

N-[2-(1,1,2,3,3,3-hexafluoropropoxy)phenyl]-2-chloronicotinamide,

N-[2-(1,1,2,3,3,3-hexafluoropropoxy)phenyl]-1-methyl-3-trifluoromethyl-1H-pyrazole-4-carboxamide,

N-[2-(1,1,2,3,3,3-hexafluoropropoxy)phenyl]-3-difluoromethyl-1-methyl-1H-pyrazole-4-carboxamide,

N-[2-(1,1,2,3,3,3-hexafluoropropoxy)phenyl]-2-methyl-4-trifluoromethylthiazole-5-carboxamide,

N-[2-(1,1,2,3,3,3-hexafluoropropoxy)phenyl]-4-difluoromethyl-2-methylthiazole-5-carboxamide,

N-[2-(2-chloro-1,1,2-trifluoroethoxy)phenyl]-1-methyl-3-trifluoromethyl-1H-pyrazole-4-carboxamide and

N-[2-(2-chloro-1,1,2-trifluoroethoxy)phenyl]-3-difluoromethyl-1-methyl-1H-pyrazole-4-carboxamide are very particularly preferred.

Compounds I according to the invention can be obtained by different routes. Advantageously, compounds I according to the invention are prepared by reacting a carbonyl halide of the formula II in the presence of a base with an aniline of the formula III (cf., for example, J. March, Advanced Organic Chemistry, 2nd Ed., 382f., McGraw-Hill 1977):

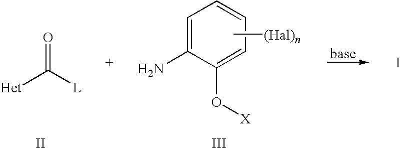

L is chlorine, bromine or iodine, in particular chlorine or bromine.

The reaction is usually carried out at temperatures of from (−20) to 100° C., preferably from 0 to 50° C., at atmospheric pressure.

Suitable solvents are aliphatic hydrocarbons, such as n-pentane, n-hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide and dimethylformamide, particularly preferably toluene and tetrahydrofuran. However, it is also possible to use mixtures of the solvents mentioned.

Suitable bases are, in general, inorganic compounds, for example alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, sodium amide and potassium amide, alkali metal and alkaline earth metal carbonates, such as lithium carbonate and calcium carbonate and also alkali metal bicarbonates, such as sodium bicarbonate, and organometallic compounds, in particular alkali metal alkyls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide, potassium tert-butoxide, and dimethoxymagnesium, moreover organic bases, for example tertiary amines, such as trimethylamine, triethylamine, triisopropylethylamine and N-methylpiperidine, pyridine, substituted pyridines, such as collidine, lutidine and 4-dimethylaminopyridine, and also bicyclic amines.

Particular preference is given to using triethylamine or pyridine.

The base is generally employed in approximately equimolar amounts, based on the carbonyl halide II. However, it can also be used in an excess of up to 30 mol %, preferably up to 10 mol %, or—if tertiary amines are used—if appropriate as solvent.

The starting materials are generally reacted with one another in about equimolar amounts. However, in terms of yield, it may also be advantageous to employ an excess of II of from 1 to 20 mol %, preferably from 0.5 to 10 mol %, based on the aniline III.

The starting materials II and III are known or can be prepared in a manner known per se (cf., for example, Helv. Chim. Acta 60, 978 (1977); Zh. Org. Chim 26, 1527 (1990); Heterocyclus 26, 1885 (1987); Izv. Akad. Nauk. SSSR Ser. Khim 1982, 2160; THL 28, 593 (1987); THL 29, 5463 (1988)).

If individual compounds I are not accessible by reaction of II with III, they can be prepared by derivatization from other compounds I.

The compounds I are suitable for use as fungicides. They are distinguished by excellent activity against a broad spectrum of phytopathogenic fungi, in particular from the classes of the Ascomycetes, Basidiomycetes, Deuteromycetes and Peronosporomycetes (syn. Oomycetes). Some of them are systemically active and can be used in crop protection as foliar fungicides, as fungicides for seed dressing and as soil fungicides.

They are particularly important in the control of a large number of fungi on various crop plants, such as wheat, rye, barley, oats, rice, corn, grass, bananas, cotton, soybeans, coffee, sugar cane, grapevines, fruit and ornamental plants and vegetables, such as cucumbers, beans, tomatoes, potatoes and cucurbits, and also the seeds of these plants.

They are especially suitable for controlling the following plant diseases:
Alternaria species on vegetables, oilseed rape, sugar beet and fruit and rice, for example, A. solani or A. alternata on potatoes and tomatoes;
Aphanomyces species on sugar beet and vegetables;
Ascochyta species on cereals and vegetables;
Bipolaris and Drechslera species on corn, cereals, rice and lawns, for example, D. maydis on corn;
Blumeria graminis (powdery mildew) on cereals;
Botrytis cinerea (gray mold) on strawberries, vegetables, flowers and grapevines;
Bremia lactucae on lettuce;
Cercospora species on corn, soybeans, rice and sugar beet;
Cochliobolus species on corn, cereals, rice, for example Cochliobolus sativus on cereals, Cochliobolus miyabeanus on rice;
Colletotricum species on soybeans and cotton;
Drechslera species, Pyrenophora species on corn, cereals, rice and lawns, for example, D. teres on barley or D. tritici-repentis on wheat;
Esca on grapevines, caused by Phaeoacremonium chlamydosporium,
Ph. Aleophilum and Formitipora punctata (syn. Phellinus punctatus);
Elsinoe ampelina on grapevines;
Exserohilum species on corn;
Erysiphe cichoracearum and Sphaerotheca fuliginea on cucumbers;
Fusarium and Verticillium species on various plants, for example, F graminearum or F. culmorum on cereals or F. oxysporum on a multitude of plants, such as, for example, tomatoes;
Gaeumanomyces graminis on cereals;
Gibberella species on cereals and rice (for example Gibberella fujikuroi on rice);
Glomerella cingulata on grapevines and other plants;
Grainstaining complex on rice;
Guignardia budwelli on grapevines;
Helminthosporium species on corn and rice;
Isariopsis clavispora on grapevines;
Michrodochium nivale on cereals;
Mycosphaerella species on cereals, bananas and groundnuts, for example, M. graminicola on wheat or M. fijiensis on bananas;
Peronospora species on cabbage and bulbous plants, for example, P. brassicae on cabbage or P. destructor on onions;
Phakopsara pachyrhizi and Phakopsara meibomiae on soybeans;
Phomopsis species on soybeans and sunflowers, P. viticola on grapevines;
Phytophthora infestans on potatoes and tomatoes;
Phytophthora species on various plants, for example, P. capsici on bell pepper;
Plasmopara viticola on grapevines;
Podosphaera leucotricha on apples;
Pseudocercosporella herpotrichoides on cereals;
Pseudoperonospora on various plants, for example, P. cubensis on cucumber or P. humili on hops;
Pseudopezicula tracheiphilai on grapevines;
Puccinia species on various plants, for example, P. triticina, P. striformins, P. hordei or P. graminis on cereals or P. asparagi on asparagus;
Pyricularia oryzae, Corticium sasakii, Sarocladium oryzae, S.attenuatum, Entyloma oryzae on rice;
Pyricularia grisea on lawns and cereals;
Pythium spp. on lawns, rice, corn, cotton, oilseed rape, sunflowers, sugar beet, vegetables and other plants, for example, P. ultiumum on various plants, P. aphanidermatum on lawns;
Rhizoctonia species on cotton, rice, potatoes, lawns, corn, oilseed rape, sugar beet, vegetables and on various plants, for example, R. solani on beet and various plants;
Rhynchosporium secalis on barley, rye and triticale;
Sclerotinia species on oilseed rape and sunflowers;
Septoria tritici and Stagonospora nodorum on wheat;
Erysiphe (syn. Uncinula) necator on grapevines;
Setospaeria species on corn and lawns;
Sphacelotheca reilinia on corn;
Thievaliopsis species on soybeans and cotton;
Tilletia species on cereals;
Ustilago species on cereals, corn and sugar cane, for example, U. maydis on corn;
Venturia species (scab) on apples and pears, for example, V. inaequalis on apples.

The compounds I are furthermore suitable for controlling harmful fungi in the protection of materials (for example wood, paper, paint dispersions, fibers or fabrics) and in the protection of stored products. In the protection of wood, particular attention is paid to the following harmful fungi: Ascomycetes, such as Ophiostoma spp., Ceratocystis spp., Aureobasidium pullulans, Sclerophoma spp., Chaetomium spp., Humicola spp., Petriella spp., Trichurus spp.; Basidiomycetes, such as Coniophora spp., Coriolus spp., Gloeophyllum spp., Lentinus spp., Pleurotus spp., Poria spp., Serpula spp. and Tyromyces spp., Deuteromycetes, such as Aspergillus spp., Cladosporium spp., Penicillium spp., Trichoderma spp., Alternaria spp., Paecilomyces spp. and Zygomycetes, such as Mucor spp., additionally in the protection of materials the following yeasts: Candida spp. and Saccharomyces cerevisae.

The compounds I are employed by treating the fungi or the plants, seeds, materials or soil to be protected from fungal attack with a fungicidally effective amount of the active compounds I. The application can be carried out both before and after the infection of the materials, plants or seeds by the fungi.

The fungicidal compositions generally comprise between 0.1 and 95%, preferably between 0.5 and 90%, by weight of active compound I.

When employed in plant protection, the amounts applied are, depending on the kind of effect desired, between 0.01 and 2.0 kg of active compound per ha.

In seed treatment, for example by dusting, coating or drenching seed, amounts of active compound of from 1 to 1000 g/100 kg, preferably from 5 to 100 g/100 kg, of seed are generally necessary.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound I per cubic meter of treated material.

The compounds I can be converted into the customary formulations, for example solutions, emulsions, suspensions, dusts, powders, pastes and granules. The use form depends on the particular intended purpose; in each case, it should ensure a fine and even distribution of the compounds I according to the invention.

The formulations are prepared in a known manner, for example by extending the active compound I with solvents and/or carriers, if desired using emulsifiers and dispersants. Solvents/auxiliaries suitable for this purpose are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (N-methylpyrrolidone, N-octylpyrrolidone), acetates (glycol diacetate), glycols, fatty acid dimethylamides, fatty acids and fatty acid esters. In principle, solvent
mixtures may also be used.
carriers such as ground natural minerals (for example kaolins, clays, talc, chalk) and ground synthetic minerals (for example highly disperse silica, silicates); emulsifiers such as nonionogenic and anionic emulsifiers (for example polyoxyethylene fatty alcohol ethers, alkylsulfonates and arylsulfonates) and dispersants such as ligno-sulfite waste liquors and methylcellulose.

Suitable surfactants used are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, highly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are for example mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers such as ammonium sulfate, ammonium phosphate, ammonium nitrate, ureas, and products of vegetable origin such as cereal meal, tree bark meal, wood meal and nutshell meal, cellulose powders and other solid carriers.

Formulations for the treatment of seed may additionally comprise binders and/or gelling agents and, if appropriate, colorants.

Binders may be added to increase the adhesion of the active compounds on the seed after the treatment. Suitable binders are, for example, EO/PO block copolymer surfactants, but also polyvinyl alcohols, polyvinylpyrrolidones, polyacrylates, polymethacrylates, polybutenes, polyisobutylenes, polystyrenes, polyethylenamines, polyethylenamides, polyethylenimines (Lupasol®, Polymin®), polyethers, polyurethanes, polyvinyl acetates, tylose and copolymers of these polymers. A suitable gelling agent is, for example, carrageen (Satiagel®).

In general, the formulations comprise from 0.01 to 95% by weight, preferably from 0.1 to 90% by weight, of the active compound I. The active compounds I are employed in a purity of from 90% to 100%, preferably 95% to 100%, (according to NMR or HPLC spectrum).

The concentrations of active compound I in the ready-for-use preparations can be varied within relatively wide ranges. In general, they are from 0.0001 to 10%, preferably from 0.01 to 1%.

The active compounds I can also be used with great success in the ultra-low volume (ULV) process, it being possible to apply formulations with more than 95% by weight of active compound or even the active compound without additives.

For the treatment of seed, the formulations in question give, after two-to-tenfold dilution, active compound concentrations of from 0.01 to 60% by weight, preferably from 0.1 to 40% by weight, in the ready-to-use preparations.

The following are examples of formulations: 1. Products for dilution with water

A) Water-Soluble Concentrates (SL)

10 parts by weight of a compound I according to the invention are dissolved in 90 parts by weight of water or in a water-soluble solvent. As an alternative, wetting agents or other auxiliaries are added. The active compound dissolves upon dilution with water. In this way, a formulation having a content of 10% by weight of active compound is obtained.

B) Dispersible Concentrates (DC)

20 parts by weight of a compound I according to the invention are dissolved in 70 parts by weight of cyclohexanone with addition of 10 parts by weight of a dispersant, for example polyvinylpyrrolidone. Dilution with water gives a dispersion. The active compound content is 20% by weight.

C) Emulsifiable Concentrates (EC)

15 parts by weight of a compound I according to the invention are dissolved in 75 parts by weight of xylene with addition of calcium dodecylbenzenesulfonate and castor oil ethoxylate (in each case 5 parts by weight). Dilution with water gives an emulsion. The formulation has an active compound content of 15% by weight.

D) Emulsions (EW, EO)

25 parts by weight of a compound I according to the invention are dissolved in 35 parts by weight of xylene with addition of calcium dodecylbenzenesulfonate and castor oil ethoxylate (in each case 5 parts by weight). This mixture is introduced into 30 parts by weight of water by means of an emulsifying machine (Ultraturrax) and made into a homogeneous emulsion. Dilution with water gives an emulsion. The formulation has an active compound content of 25% by weight.

E) Suspensions (SC, OD)

In an agitated ball mill, 20 parts by weight of a compound I according to the invention are comminuted with addition of 10 parts by weight of dispersants and wetting agents and 70 parts by weight of water or an organic solvent to give a fine active compound suspension. Dilution with water gives a stable suspension of the active compound. The active compound content in the formulation is 20% by weight.

F) Water-Dispersible Granules and Water-Soluble granules (WG, SG)

50 parts by weight of a compound I according to the invention are ground finely with addition of 50 parts by weight of dispersants and wetting agents and prepared as water-dispersible or water-soluble granules by means of technical appliances (for example extrusion, spray tower, fluidized bed). Dilution with water gives a stable dispersion or solution of the active compound. The formulation has an active compound content of 50% by weight.

G) Water-Dispersible Powders and Water-Soluble Powders (WP, SP)

75 parts by weight of a compound I according to the invention are ground in a rotor-stator mill with addition of 25 parts by weight of dispersants, wetting agents and silica gel. Dilution with water gives a stable dispersion or solution of the active compound. The active compound content of the formulation is 75% by weight.

2. Products to be Applied Undiluted

H) Dustable Powders (DP)

5 parts by weight of a compound I according to the invention are ground finely and mixed intimately with 95 parts by weight of finely divided kaolin. This gives a dustable product having an active compound content of 5% by weight.

J) Granules (GR, FG, GG, MG)

0.5 part by weight of a compound I according to the invention is ground finely and associated with 99.5 parts by weight of carriers. Current methods are extrusion, spray-drying or the fluidized bed. This gives granules to be applied undiluted having an active compound content of 0.5% by weight.

K) ULV Solutions (UL)

10 parts by weight of a compound I according to the invention are dissolved in 90 parts by weight of an organic solvent, for example xylene. This gives a product to be applied undiluted having an active compound content of 10% by weight.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the active compounds I according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetting agent, tackifier, dispersant or emulsifier. However, it is also possible to prepare concentrates composed of active substance, wetting agent, tackifier, dispersant or emulsifier and, if appropriate, solvent or oil, and such concentrates are suitable for dilution with water.

Oils of various type, wetting agents, adjuvants, herbicides, fungicides, other pesticides, or bactericides may be added to the active compounds, even, if appropriate, not until immediately prior to use (tank mix). These agents may be admixed with the compositions according to the invention in a weight ratio of from 1:100 to 100:1, preferably from 1:10 to 10:1.

Suitable adjuvants in this sense are in particular: organically modified polysiloxanes, for example Break Thru S 240®; alcohol alkoxylates, for example Atplus 245®, Atplus MBA 1303®, Plurafac LF 300® and Lutensol ON 30®; EO/PO block polymers, for example Pluronic RPE 2035® and Genapol B®; alcohol ethoxylates, for example Lutensol XP 80®; and sodium dioctylsulfosuccinate, for example Leophen RA®.

The N-[2-(haloalkoxy)phenyl]heteroarylcarboxamides I according to the invention can also be used together with other active compounds, for example with herbicides, insecticides, growth regulators or with fertilizers.

### PREPARATION EXAMPLES

The procedures described in the following synthesis examples were used to prepare further compounds I by appropriate modification of the starting materials. The compounds I obtained in this manner are listed in Table 4 below, together with physical data.

### Example 1

### Synthesis of N-[2-(1,1,2,2-tetrafluoroethoxy)phenyl]-3-difluoromethyl-1-methyl-1H-pyrazole-4-carboxamide

At about 20° C., 0.23 g of 3-difluoromethyl-1-methyl-1H-pyrazole-4-carbonyl chloride was added dropwise to a solution of 0.25 g of 2-(1,1,2,2-tetrafluoroethoxy)phenylamine and 0.14 g of pyridine in 8.5 ml of tetrahydrofuran, and the mixture was then stirred at about 20° C. for 16 hours. 40 ml of methyl tert-butyl ether were then added, and the organic phase was washed successively with 2% strength hydrochloric acid, twice with 2% strength aqueous sodium hydroxide solution and with dilute aqueous sodium chloride solution. The organic phase was dried and concentrated under reduced pressure. Diisopropyl ether was added to the crude product, and the undissolved solid was then separated off, washed with pentane and dried. Yield: 0.43 g of product of value as a white powder; m.p. 117-119° C.

### Example 2

### N-[2-(2-Chloro-1,1,2-trifluoroethoxy)phenyl]-1-methyl-3-trifluoromethyl-1H-pyrazole-4-carboxamide

At room temperature, 0.28 g of 3-trifluoromethyl-1-methyl-1H-pyrazole-4-carbonyl chloride were added dropwise to a soultion of 0.29 g of 2-(2-chloro-1,1,2-trifluoroethoxy)-phenylamine and 0.15 g of pyridine in 10 ml of toluene. The mixture was then stirred at about 20° C. for 16 hours. 30 ml of methyl tert-butyl ether were then added, and the organic phase was washed successively with 2% strength hydrochloric acid, twice with 2% strength aqueous sodium hydroxide solution and with dilute aqueous sodium chloride solution. The organic phase was dried and concentrated under reduced pressure. The crude product was purified by column chromatography on silica gel using a mixture of toluene/methyl tert-butyl ether/cyclohexane (1:1:1). Yield: 0.37 g of product of value as a white powder; m.p. 94-95° C.

### Example 3

### N-[2-(1,1,2,3,3,3-Hexafluoropropoxy)phenyl]-1-methyl-3-trifluoromethyl-1H-pyrazole-4-carboxamide

At room temperature, 0.50 g of 2-(1,1,2,3,3,3-hexafluoropropoxy)phenylamine and 0.86 g of bis(2-oxo-3-oxazolidinyl)phosphoryl chloride were added to a solution of 0.43 g of 1-methyl-3-trifluoromethyl-1H-pyrazole-4-carboxylic acid and 0.45 g of triethylamine in 30 ml of dichlormethane. The mixture was stirred at about 20° C. for 60 hours and then washed successively twice with dilute hydrochloric acid, twice with aqueous sodium bicarbonate solution and once with water. The organic phase was dried and concentrated. The crude product was purified by column chromatography (mobile phase: cyclohexane/methyl tert-butyl ether=1:2) on silica gel. Yield: 0.35 g of product of value as a clear oil.

TABLE 4
Compounds I where n = 0

| | | | | | | | Phys. data |
|---|---|---|---|---|---|---|---|
| No. | Het | R¹ | R² | R³ | R⁴ | X | (m.p. [° C.]) |
| I-1 | (a) | CF₃ | H | — | — | —CF₂—CHF—CF₃ | 91-93 |
| I-2 | (a) | CHF₂ | H | — | — | —CF₂—CHF—CF₃ | 97-99 |
| I-3 | (a) | CF₃ | H | — | — | —CF₂—CHF—Cl | 94-95 |
| I-4 | (a) | CHF₂ | H | — | — | —CF₂—CHF—Cl | 110-111 |
| I-5 | (a) | CHF₂ | H | — | — | —CF₂—CHF₂ | 117-119 |
| I-6 | (b) | — | — | CF₃ | — | —CF₂—CHF₂ | 118-120 |
| I-7 | (b) | — | — | CH₃ | — | —CF₂—CHF₂ | 134-135 |
| I-8 | (b) | — | — | CF₃ | — | —CF₂—CHF—CF₃ | 70-71 |
| I-9 | (b) | — | — | CF₃ | — | —CF₂—CHF—Cl | 80-81 |
| I-10 | (b) | — | — | CHF—CH₃ | — | —CF₂—CHF₂ | 114-115 |
| I-11 | (b) | — | — | CHF₂ | — | —CF₂—CHF—CF₃ | 89-92 |
| I-12 | (c) | — | — | — | Cl | —CF₂—CHF₂ | 126-128 |
| I-13 | (c) | — | — | — | Cl | —CF₂—CHF—CF₃ | 85-91 |
| I-14 | (c) | — | — | — | Cl | —CF₂—CHF—Cl | 73-76 |

### Use Examples

The fungicidal effect of the compounds 1 according to the invention was demonstrated by the following tests:

The active compounds were separately or jointly prepared as a stock solution comprising 25 mg of active compound which was made up to 10 ml using a mixture of acetone and/or dimethylsulfoxide and the emulsifier Uniperol® EL (wetting agent having emulsifying and dispersing action based on ethoxylated alkylphenols) in a volume ratio of solvent/emulsifier of 99 to 1. The mixture was then made up with water to 100 ml. This stock solution was diluted with the solvent/emulsifier/water mixture described to the desired concentration of active compounds.

### Use Example 1

### Activity Against Late Blight on Tomatoes Caused by Phytophthora infestans, 1 Day Protective Treatment

Leaves of potted tomato plants were sprayed to runoff point with an aqueous suspension comprising 63 ppm or 250 ppm of active compound. 1, 3 or 5 days after the application, the leaves were infected with an aqueous sporangia suspension of Phytophthora infestans. The plants were then placed in a water vapor-saturated chamber at temperatures between 18 and 20° C. After 6 days, the late blight on the untreated, but infected control plants had developed to such an extent that the infection could be determined visually in %.

### Use Example 2

### Activity Against Early Blight on tomatoes Caused by Alternaria solani

Leaves of potted tomato plants of the cultivar “Goldene Königin” were sprayed to runoff point with an aqueous suspension having the concentration of active compounds stated below. The next day, the leaves were infected with an aqueous spore suspension of Alternaria solani in a 2% biomalt solution having a density of 0.17×10⁶ spores/ml. The plants were then placed in a water vapor-saturated chamber at temperatures between 20 and 22° C. After 5 days, the disease on the untreated, but infected control plants had developed to such an extent that the infection could be determined visually in %.

In this test, the plants which had been treated with 250 ppm of the compounds nos. I-1, I-2, I-3, I-4 and I-5 showed an infection of not more than 30%.

By contrast, the plants treated with 250 ppm of comparative compound N-[2-(1,1,2,2-tetrafluoroethoxy)phenyl]-1,3-dimethylpyrazol-4-yl-carboxamide showed an infection of 60%. The untreated plants were 90% infected.

### Use Example 3

### Activity Against Mildew of Wheat Caused by Erysiphe [syn. Blumeria] graminis forma specialis. tritici

Leaves of potted wheat seedlings were sprayed to runoff point with an aqueous suspension having the active compound concentration stated below. The suspension was prepared as described above. 24 hours after the spray coating had dried on, the plants were dusted with spores of mildew of wheat (Erysiphe [syn. Blumeria] graminis forma specialis. tritici). The test plants were then placed in a greenhouse at temperatures between 20 and 24° C. and at 60 to 90% relative atmospheric humidity. After 7 days, the extent of the mildew development was determined visually in % infection of the entire leaf area.

In this test, the plants which had been treated with 63 ppm of the compounds nos. I-1, I-2, I-3, I-4 and I-5 showed an infection of not more than 40%.

By contrast, the plants treated with 63 ppm of the comparative compound N-[2-(1,1,2,2-tetrafluoroethoxy)phenyl]-3-trifluormethyl-1-methylpyrazol-4-yl-carboxamide showed an infection of 70%. The untreated plants were 90% infected.

These plants which had been treated with 250 ppm of the compounds nos. I-1, I-2, I-3, I-4 and I-5 showed an infection of not more than 20%.

By contrast, the plants treated with 250 ppm of the comparative compound N142-(1,1,2,2-tetrafluoroethoxy)phenyl]-3-trifluormethyl-1-methylpyrazol-4-yl-carboxamide showed an infection of 40%. The untreated plants were 80% infected.

### Use Example 4

### Activity Against Gray Mold on Bell Pepper Leaves Caused by Botrytis cinerea, Protective Application

Bell pepper seedlings of the cultivar “Neusiedler Ideal Elite” were, after 2-3 leaves were well developed, sprayed to runoff point with an aqueous suspension in the active compound concentration stated below. The next day, the treated plants were inoculated with a spore suspension of Botrytis cinerea which comprised 1.7×10⁶ spores/ml in a 2% strength aqueous biomalt solution. The test plants were then placed in a dark climatized chamber at 22 to 24° C. and high atmospheric humidity. After 5 days, the extent of the fungal infection on the leaves could be determined visually in %.

In this test, the plants which had been treated with 250 ppm of the compounds nos. I-1, I-2, I-3, I-4, I-5, I-6, I-8, I-9, I-11, I-12, I-13 and I-14 showed an infection of not more than 20%, whereas the untreated plants were 75% infected.

### Use Example 5

### Curative Activity Against Brown Rust of Wheat Caused by Puccinia recondita

Leaves of potted wheat seedlings of the cultivar “Kanzler” were inoculated with a spore suspension of brown rust (Puccinia recondita). The pots were then placed in a chamber with high atmospheric humidity (90 to 95%) and at 20 to 22° C. for 24 hours. During this time, the spores germinated and the germ tubes penetrated into the leaf tissue. The next day, the infected plants were sprayed to runoff point with the above-described active compound solution at the active compound concentration stated below. After the spray coating had dried on, the test plants were cultivated in a greenhouse at temperatures between 20 and 22° C. and at 65 to 70% relative atmospheric humidity for 7 days. The extent of the rust fungus development on the leaves was then determined.

In this test, the plants which had been treated with 250 ppm of the compounds nos. I-1, I-2, I-3, I-4, I-5, I-6, I-7, I-8, I-9, I-11, I-12, I-13 and I-1 than 15%, whereas the untreated plants were 90% infected.

1. An N-[2-(haloalkoxy)phenyl]heteroarylcarboxamide of the formula I 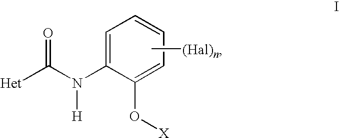
in which the variables are as defined below:
n is 0 or 1;
Hal is halogen;
X is C₂-C₄-haloalkyl;
Het is a pyrazole, thiazole or pyridine radical of the formula (a), (b) or (c) 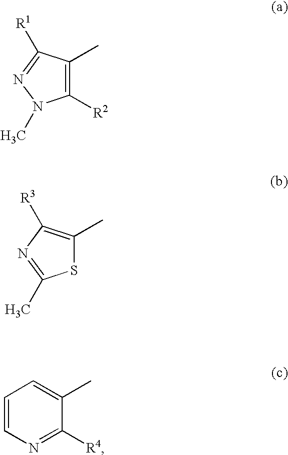
where
R¹ is C₁-C₄-alkyl or C₁-C₄-haloalkyl,
R² is hydrogen or halogen,
R³ is C₁-C₄-alkyl or C₁-C₄-haloalkyl and
R⁴ is halogen, C₁-C₄-alkyl or C₁-C₄-haloalkyl,
except for N-[2-(1,1,2,2-tetrafluoroethoxy)phenyl]-1,3-dimethylpyrazol-4-yl-carboxamide, N-[2-(1,1,2,2-tetrafluoroethoxy)phenyl]-3-trifluoromethyl-1-methyl-pyrazol-4-ylcarboxamide and N-[2-(2,2,2-trifluorethoxy)phenyl]-3-trifluormethyl-1-methylpyrazol-4-yl-carboxamide.

2. The N-[2-(haloalkoxy)phenyl]heteroarylcarboxamide of the formula I according to claim 1 where n is 0.

3. The N-[2-(haloalkoxy)phenyl]heteroarylcarboxamide of the formula I according to claim 1 where X is n-propyl or isopropyl which carries in each case one to 7 halogen atoms selected from 7 fluorine atoms and 1 chlorine atom.

4. The N-[2-(haloalkoxy)phenyl]heteroarylcarboxamide of the formula I according to claim 1 where X is 1,1,2,3,3,3-hexafluoropropyl.

5. The N-[2-(haloalkoxy)phenyl]heteroarylcarboxamide of the formula I according to claim 1 where X is 2-chloro-1,1,2-trifluoroethyl.

6. The N-[2-(haloalkoxy)phenyl]heteroarylcarboxamide of the formula I according to claim 1 where R¹ is difluoromethyl and X is 1,1,2,2-tetrafluoroethyl.

7. The N-[2-(haloalkoxy)phenyl]heteroarylcarboxamide of the formula I according to claim 1, selected from the following group:
N-[2-(1,1,2,2-tetrafluoroethoxy)phenyl]-3-difluoromethyl-1-methylpyrazol-4-yl-carboxamide,
N-[2-(1,1,2,2-tetrafluoroethoxy)phenyl]-1,3-dimethyl-5-fluoropyrazol-4-yl-carboxamide,
N-[2-(1,1,2,2-tetrafluoroethoxy)phenyl]-3-difluoromethyl-5-fluoro-1-methylpyrazol-4-ylcarboxamide,
N-[2-(1,1,2,2-tetrafluoroethoxy)phenyl]-5-fluoro-1-methyl-3-trifluoromethylpyrazol-4-ylcarboxamide,
N-[2-(1,1,2,2-tetrafluoroethoxy)phenyl]-5-chloro-1,3-dimethylpyrazol-5-chloro-4-ylcarboxamide,
N-[2-(1,1,2,2-tetrafluoroethoxy)phenyl]-5-chloro-3-difluoromethyl-1-methylpyrazol-4-ylcarboxamide,
N-[2-(1,1,2,2-tetrafluoroethoxy)phenyl]-5-chloro-1-methyl-3-trifluoromethyl-pyrazol-4-ylcarboxamide,
N-[2-(1,1,2,2-tetrafluoroethoxy)phenyl]-2,4-dimethylthiazol-5-ylcarboxamide,
N-[2-(1,1,2,2-tetrafluoroethoxy)phenyl]-4-difluoromethyl-2-methylthiazol-5-yl-carboxamide,
N-[2-(1,1,2,2-tetrafluoroethoxy)phenyl]-2-methyl-4-trifluoromethylthiazol-5-yl-carboxamide,
N-[2-(1,1,2,2-tetrafluoroethoxy)phenyl]-2-methylnicotinamide,
N-[2-(1,1,2,2-tetrafluoroethoxy)phenyl]-2-trifluoromethylnicotinamide,
N-[2-(1,1,2,2-tetrafluoroethoxy)phenyl]-2-fluoronicotinamide or
N-[2-(1,1,2,2-tetrafluoroethoxy)phenyl]-2-chloronicotinamide,
N-[2-(2-chloro-1,1,2-trifluoroethoxy)phenyl]-2-chloronicotinamide,
N-[2-(2-chloro-1,1,2-trifluoroethoxy)phenyl]-2-methyl-4-trifluoromethylthiazole-5-carboxamide,
N-[2-(1,1,2,2-tetrafluoroethoxy)phenyl]-3-(chlorofluoromethyl)-1-methyl-1H-pyrazole-4-carboxamide,
N-[2-(1,1,2,2-tetrafluoroethoxy)phenyl]-3-fluoromethyl-1-methyl-1H-pyrazole-4-carboxamide,
N-[2-(1,1,2,2-tetrafluoroethoxy)phenyl]-3-(dichlorofluoromethyl)-1-methyl-1H-pyrazole-4-carboxamide,
N-[2-(1,1,2,3,3,3-hexafluoropropoxy)phenyl]-2-chloronicotinamide,
N-[2-(1,1,2,3,3,3-hexafluoropropoxy)phenyl]-1-methyl-3-trifluoromethyl-1H-pyrazole-4-carboxamide,
N-[2-(1,1,2,3,3,3-hexafluoropropoxy)phenyl]-3-difluoromethyl-1-methyl-1H-pyrazole-4-carboxamide,
N-[2-(1,1,2,3,3,3-hexafluoropropoxy)phenyl]-2-methyl-4-trifluoromethylthiazole-5-carboxamide,
N-[2-(1,1,2,3,3,3-hexafluoropropoxy)phenyl]-4-difluoromethyl-2-methylthiazole-5-carboxamide,
N-[2-(2-chloro-1,1,2-trifluoroethoxy)phenyl]-1-methyl-3-trifluoromethyl-1H-pyrazole-4-carboxamide and
N-[2-(2-chloro-1,1,2-trifluoroethoxy)phenyl]-3-difluoromethyl-1-methyl-1 H-pyrazole-4-carboxamide.

8. A fungicidal composition comprising at least one liquid or solid carrier and at least one compound of the formula I according to claim 1.

9. The fungicidal composition according to claim 8, additionally comprising a further active compound.

10. The use of the compounds I according to claim 1 for preparing a composition suitable for controlling harmful fungi.

11. A method for controlling phytopathogenic harmful fungi, which method comprises treating the fungi, their habitat or the plants, the soil, seed, areas, materials or spaces to be protected from fungal attack with an effective amount of at least one compound I according to claim 1.

12. Seed, comprising at least one N-[2-(haloalkoxy)phenyl]heteroarylcarboxamide of the formula I according to claim 1 in an amount of from 1 to 1000 g per 100 kg.
